(19) United States
(12) Patent Application Publication
(10) Pub. No.: US 2009/0254076 A1
(43) Pub. Date: Oct. 8, 2009

(54) METHOD AND APPARATUS FOR FRACTIONAL DEFORMATION AND TREATMENT OF TISSUE

(75) Inventors: Gregory B. Altshuler, Lincoln, MA (US); Andrei Belikov, St. Petersburg (RU); James Childs, Bolton, MA (US); Andrei Erofeev, North Andover, MA (US); Ilya Yaroslavsky, North Andover, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: PALOMAR MEDICAL CORPORATION, Burlington, MA (US)

(21) Appl. No.: 12/405,931

(22) Filed: Mar. 17, 2009

Related U.S. Application Data
(60) Provisional application No. 61/069,678, filed on Mar. 17, 2008.
(60) Provisional application No. 61/188,339, filed on Aug. 8, 2008.
(60) Provisional application No. 61/198,272, filed on Nov. 3, 2008.

(51) Int. Cl. A61B 18/18

(52) U.S. Cl. 606/33

(57) ABSTRACT

Devices and methods of treatment of tissue, such as skin tissue, with electromagnetic radiation (EMR) are disclosed that employ local deformation of tissue in small areas. Devices and methods employing local deformation are used to produce fractional lattices of EMR-treated islets in tissue.

### RELATED APPLICATIONS

This application claims the benefit of and priority to and is the non provisional application of U.S. Ser. No. 61/069,678 filed Mar. 17, 2008 entitled “Method and Apparatus for Fractional Deformation and Treatment of Tissue.” This application also claims the benefit of and priority to and is the non provisional application of U.S. Ser. No. 61/188,339 filed Aug. 8, 2008 entitled “Method and Apparatus for Fractional Deformation and Treatment of Tissue.” This application also claims the benefit of and priority to and is the non provisional application of U.S. Ser. No. 61/198,272 filed Nov. 3, 2008 entitled “Combined Fractional Ablative and Fractional Non-Ablative Treatment.”

### BACKGROUND

1. Field of the Invention

The devices and methods disclosed herein relate to the treatment of soft and hard tissues with electromagnetic energy generally, including, without limitation, optical energy having wavelengths in the ultraviolet, visible and infrared ranges. More specifically, the devices and methods disclosed herein relate to the treatment of tissue with electromagnetic radiation (EMR) in conjunction with local deformation of tissue in small areas. Also disclosed are devices and systems for producing lattices of EMR-treated islets in tissue, and cosmetic and medical applications of such devices and systems.

2. Description of the Related Art

Electromagnetic radiation, particularly in the form of laser light, has been used in a variety of cosmetic and medical applications, including uses in dermatology, dentistry, ophthalmology, gynecology, otorhinolaryngology and internal medicine. For most dermatological applications, the EMR treatment can be performed with a device that delivers the EMR to the surface of the targeted tissues. For applications in internal medicine, the EMR treatment is typically performed with a device that works in combination with an endoscope or catheter to deliver the EMR to internal surfaces and tissues.

As a general matter, existing EMR treatments are typically designed to (a) deliver one or more particular wavelengths (or a range (or ranges) of wavelengths) of EMR to a tissue to induce a particular chemical reaction, (b) deliver EMR energy to a tissue to cause an increase in temperature, or (c) deliver EMR energy to a tissue to damage or destroy cellular or extra cellular structures, such as for skin remodeling.

For skin remodeling, absorption of optical energy by water is widely used in two approaches: ablative skin resurfacing, typically performed with either CO₂ (10.6 μm) or Er:YAG (2.94 μm) lasers, and non-ablative skin remodeling using a combination of deep skin heating with light from Nd:YAG (1.34 μm), Er:glass (1.56 μm) or diode laser (1.44 μm) and skin surface cooling for selective damage of sub-epidermal tissue. Ablative skin resurfacing generally employs a wavelength range of from about 2600 nm to about 11000 nm. Non-ablative skin treatments generally employ a wavelength range of from about 290 nm to about 2600 nm. Non-ablative techniques offer considerably reduced risk of side effects and are much less demanding on post-operative care. However, clinical efficacy of the non-ablative procedures has not been satisfactory.

In the cosmetic field for the treatment of various skin conditions, alternative methods and devices have been developed that irradiate or cause damage in a portion of the tissue area and/or volume being treated. These methods and devices have become known as fractional technology. Fractional technology is thought to be a safer method of treatment of skin for cosmetic purposes, because tissue damage occurs within smaller sub-volumes or islets within the larger volume of tissue being treated. The tissue surrounding the “treated” and/or “damaged” islets is spared from the damage. Because the resulting islets are surrounded by neighboring healthy tissue (i.e., neighboring untreated tissue) the healing process is thorough and fast. Furthermore, it is believed that the surrounding healthy tissue aids in healing and the treatment effects of the damaged tissue.

Examples of devices that have been used to treat the skin using non-ablative procedures such as skin resurfacing include the Palomar® 1540 Fractional Handpiece, the Reliant Fraxel® SR Laser and similar devices by ActiveFX, Alma Lasers, Iridex, and Reliant Technologies. Examples of devices that have been used to treat the skin using ablative procedures include the Palomar® 2940 Fractional Handpiece, for example. Methods and devices for fractional non-ablative and fractional ablative treatment are disclosed in US Patent Publication No. US2008/0172047A1, U.S. Ser. No. 11/966,468, filed Dec. 28, 2007; in U.S. Pat. No. 6,997,923, U.S. Ser. No. 10/033,302, filed Dec. 27, 2001 and related family member applications, and the contents of all of these applications are incorporated by reference herein.

Although fractional technology is presently thought to be superior to other EMR-based technologies for many applications, the fractional application of EMR can be made more efficient and effective.

### SUMMARY

For example, in many applications, the depth of penetration of the fractional columns into the tissue is believed to be important to the effectiveness of the treatment. In applications where depth of penetration into human skin is important, a deeper column capable of reaching deeper into the dermis of the tissue or even to the hypodermis will result in greater effectiveness of the treatment. However, fractional columns have generally been made deeper by applying more energy, which has other ramifications, including cost of the device and the application of more power to the tissue which can result in more damage (e.g., collateral damage, unintended damage and/or undesirable damage) to the tissue and the diffusion of additional heat within the tissue. Similarly, in other applications, a shallower depth that can be achieved using less energy will allow a device to use less energy, a less costly EMR source and/or the application of less energy per volume of tissue.

By developing additional devices and methods to more efficiently deliver EMR in a fractional treatment, the fractional devices and treatments can be further optimized and improved. For example, deeper treatment columns can be created, less costly light sources could be used, more energy efficient devices could be created, zones of damage could be created at the same depth using less energy per unit of volume, and/or more effective treatments could be created. The present disclosure depends, in part, upon the discovery that, by deforming tissue in a small area, the tissue can be treated more effectively and/or that a device or treatment can be more efficient or otherwise optimized. In particular, when a small area of tissue is deformed by applying pressure to the area and a beam of EMR is applied to the deformed area, the penetration of the EMR into the tissue is greater than the penetration of the same beam of EMR into tissue that is not so deformed. More specifically, the depth of the damage from the EMR beam applied to deformed tissue is deeper than the depth of the damage from the EMR beam when applied to relaxed tissue (tissue that was not deformed of that is no longer deformed). This phenomenon can be used, in particular, to improve existing fractional treatments of tissue with EMR and to develop new such treatments. However, the principle is also applicable to non-fractional treatments, where the deformation of a number of small areas of tissue can be used to improve the penetration of the effect of EMR in non-fractional applications that treat a relatively larger area relative to the size deformed areas.

Downtime is the time associated with a procedure in which a patient can not or chooses to not resume normal activities as a result of the procedure. Fractional non-ablative techniques require little to no downtime relative to Fractional ablative techniques which results in an appearance (e.g., bleeding and/or oozing) that can require a few days of downtime. However, fractional non-ablative techniques generally require multiple treatments to achieve clinically desirable outcomes. In one aspect, the disclosure relates to an apparatus for performing a treatment on a tissue (e.g., skin, subcutaneous tissue such as fat and/or muscle). A treatment device has an electromagnetic radiation source and is configured to deliver electromagnetic radiation. The apparatus includes an applicator (e.g., a deformation application) for creating point compression of tissue. The electromagnetic radiation source is in communication with the applicator, which has one or more point compression elements. The one or more point compression elements each has a contact compression surface. The applicator is configured to deliver an electromagnetic radiation beam into the tissue through the contact compression surface during operation. The contact compression surface has a size larger than the electromagnetic radiation beam delivered there through. In one embodiment, at least a portion of the applicator is cooled (e.g., to below external skin temperature and/or to below tissue temperature). Alternatively or in addition, at least a portion of the one or more point compression elements is cooled. At least a portion of the one or more point compression elements may be transparent to electromagnetic radiation (e.g., made from a transparent material such as sapphire).

Each contact compression surface of the point compression element(s) has an outer perimeter and an inner perimeter. In one embodiment of the apparatus, the applicator is configured to deliver the electromagnetic radiation beam solely through an inner perimeter of the contact compression surface. In some embodiments, the one or more point compression elements form an array and the distance between adjacent point compression elements is substantially constant. Optionally, the portion of one contact compression surface that delivers the electromagnetic radiation beam is a substantially constant distance from the portion of an adjacent contact compression surface that delivers the electromagnetic radiation beam. In some embodiments, the applicator is configured to deliver the electromagnetic radiation beam through the contact compression surface such that the distance between adjacent electromagnetic radiation beams is substantially constant. In addition, the distance between adjacent point compression elements may be substantially constant.

In some embodiments, at least one dimension of the contact compression surface of the one or more point compression element(s) is less than about 3 mm. In some embodiments, the distance between adjacent point compression elements is greater than the two times smallest dimension of the contact compression surface. Optionally, the distance between adjacent point compression elements is greater than the three times smallest dimension of the contact compression surface. In one embodiment, the point compression element is a cylindrical groove and the smallest dimension of the contact compression surface can be several order of magnitude than the largest dimension of the cylindrical grooves contact compression surface (e.g., the surface that forms the line-like appearance). In some embodiments, at least one dimension of the contact compression surface is no more than twice the desired depth of tissue treatment.

In one embodiment, the one or more point compression elements each have a collocated electromagnetic radiation beam. When an electromagnetic beam is collocated in a point compression element it is delivered through the center of the point compression element. Where an electromagnetic beam is collocated in a contact compression surface of a point compression element, the electromagnetic beam is delivered through the center of the contact compression surface. In some embodiments, the distance between adjacent electromagnetic radiation beams (e.g., collocated electromagnetic radiation beams) is substantially the same.

In some embodiments, the apparatus (e.g., the applicator) includes a mechanical implement that applies pressure to the one or more point compression elements. The applied pressure can be up to about 50 Newtons/cm², for example. In another embodiment, the apparatus (e.g., the applicator) also includes a transducer that provides a signal when a desired compression of tissue is reached. The signal can be, for example, audio (e.g., a buzzing or other sound indicating a certain compression has been achieved), and/or visual (e.g., a light or a dial of measurement including the compression level that has been achieved). The transducer can be mechanical, electromechanical, and/or electrical, for example. In some embodiments, the desired compression of tissue is up to 50 Newtons/cm² and at this level the signal is provided to the user.

In some embodiments, the apparatus has a source of electromagnetic radiation with a wavelength range of from about 290 nm to about 11000 nm, a fluence of from about 0.1 J/cm² to about 1000 J/cm², a pulse width of from about 1 nanosecond to continuous wave, and a radiation beam with a spot size of from about 50 microns to about 3 mm.

In another aspect, the disclosure relates to a method for treating a region of tissue (e.g., skin, subcutaneous tissue such as fat and/or muscle). The method includes pressing a contact compression surface of one or more point compression elements to a skin surface and applying electromagnetic radiation through the one or more contact compression surfaces and each of the one or more contact compression surfaces has a size larger than the size of the electromagnetic radiation beam delivered there through. In one embodiment of the method, the electromagnetic radiation beam causes at least one of thermal, chemical, and mechanical effects on the region of tissue. In another embodiment, the method includes applying two or more pulses of electromagnetic radiation without removing the one or more point compression elements from the skin surface between applied pulses (i.e., stacking of pulses).

In another embodiment of the method, the contact compression surface of one or more point compression elements is pressed to a skin surface to displace tissue fluid from the compressed tissue (e.g., the tissue compressed under the contact compression surface of the point compression element). The tissue fluid can include at least one of water, blood, and lymph.

In another embodiment of the method, electromagnetic radiation is applied through the one or more contact compression surfaces, wherein each of the one or more contact compression surfaces has a size larger than the size of the electromagnetic radiation beam delivered there through and wherein the electromagnetic radiation is delivered to at least a portion of the compressed tissue where tissue fluid is displaced due to compression by the contact compression surface of the point compression element. In some embodiments, the contact compression surface of one or more point compression elements is pressed to a skin surface for at least 1 second or for at least three seconds. The length of time that the point compression element is pressed to the skin surface can depend on the size of the point compression element and more particularly the size of the contact compression point, with a smaller size requiring less time for fluid displacement than a larger size pressed on the same tissue region.

In some embodiments, the method includes applying electromagnetic radiation through the one or more contact compression surfaces, the electromagnetic radiation source is in communication with an applicator, and the one or more point compression elements are disposed on the applicator, wherein each of the one or more contact compression surfaces has a size larger than the size of the electromagnetic radiation beam delivered there through. The method can also include cooling at least a portion of the skin surface (e.g., the tissue and/or of the subcutaneous tissue such as fat and/or muscle) with one or more point compression elements. In other embodiments, the method also includes cooling at least a portion of the skin surface with the applicator.

In some embodiments, at least one dimension of the one or more contact compression surface is no more than three times greater than the desired depth of tissue treatment (the tissue being treated can include skin, and subcutaneous tissue such as fat and/or muscle). In some embodiments, the treatment depth ranges from about 0.1 mm to about 10 mm or from about 0.1 mm to about 3 mm. In one embodiment, the surface area of the one or more contact compression surfaces is no more than 30% of the surface of the region of tissue being treated. It is desirable that the region of tissue being treated not be clustered together and rather be dispersed over the region of tissue. In some embodiments, at least one dimension of the one or more contact compression surface is no more than the desired depth of tissue treatment.

In some embodiments, at least a portion of each of the one or more point compression elements is transparent to electromagnetic radiation (e.g., made from a transparent material such as sapphire).

In some embodiments, the one or more point compression elements form an array and the distance between the centers of adjacent point compression elements is substantially constant and electromagnetic radiation is applied through the center of the one or more contact compression surfaces such that the distance between adjacent electromagnetic radiation beams is substantially constant. In addition each of the one or more contact compression surfaces has a size larger than the size of the electromagnetic radiation beams delivered there through. In some embodiments, the one or more point compression elements each has a collocated electromagnetic radiation beams. Optionally, the distance between adjacent electromagnetic radiation beams is substantially the same.

In one embodiment, the method employs electromagnetic radiation with a wavelength range of from about 290 nm to about 11000 nm, a fluence of from about 0.1 J/cm2 to about 1000 J/cm2, a pulse width of from about 1 nanosecond to continuous wave, and a radiation beam with a spot size of from about 50 microns to about 3 mm. The one or more point compression elements can have a depth of deformation (into the region of tissue in which they are pressed) that ranges from about 100 microns to about 3 mm at the deepest point of deformation relative to the normal surface of the skin surface. In some embodiments, the method includes receiving a signal when a desired compression is reached. The desired compression is up to 50 Newtons/cm².

In another aspect, the disclosure relates to a method for treating a volume of a patient's skin by irradiating portions of the volume. The method includes providing a first source for generating non-ablative treatment radiation and delivering a non-ablative treatment radiation to selected treatment regions in the volume to a depth of up to 2 mm such that following application of the non-ablative treatment the ratio of the non-ablative treatment regions to the volume is from about 1% to about 75%. The first source for generating non-ablative treatment radiation can have a wavelength of from about 900 nm to about 2600 nm. The method also includes providing a second source for generating ablative treatment radiation and delivering, subsequent to the non-ablative treatment radiation, an ablative treatment radiation to selected treatment regions in the volume to a depth of up to 400 microns such that following application of the ablative treatment the ratio of the ablative treatment regions to the volume is from about 1% to about 75% and wherein both the non-ablative treatment regions and the ablative treatment regions are separated by untreated tissue. The second source for generating ablative treatment radiation can have a wavelength of from about 2600 to about 11000. The method can optionally include pressing a contact compression surface of one or more point compression elements to a skin surface and applying at least one of the non-ablative treatment radiation and the ablative treatment radiation through the one or more contact compression surfaces. In one embodiment, the volume of non-ablative treatment is greater than the volume of ablative treatment.

In another aspect, the disclosure relates to a method for treating a volume of a patient's skin by irradiating portions of the volume. In accordance with the method a single source for generating non-ablative treatment radiation and ablative treatment radiation is provided. Both non-ablative treatment radiation and ablative treatment radiation are delivered in a first selected treatment region within said volume such that following application of both non-ablative treatment radiation and ablative treatment radiation the first treatment region is separated by untreated tissue within said volume. In some embodiments, the pulses of non-ablative and ablative treatment radiation are stacked in the first selected treatment region.

Devices and methods of producing islets and/or islands and/or columns of treatment (e.g., damage) are disclosed. Such treatments can permit various therapeutic treatments on a patient's body at depths up to approximately 4 mm. Formation of islands and/or columns of damage in three dimensions facilitates healing (by permitting continued blood flow and cell proliferation between skin layers and islands of damage and in the untreated regions of a volume of treated tissue). In this way patient discomfort may be reduced. In addition, the fractional approach permits targeting of specific components for treatment without damage to surrounding parts of the patient's body, thereby more efficiently using the applied radiation while also reducing peripheral damage to the patient's body as the result of such treatment. The wavelengths utilized for treatment can be selected for the desired depth of treatment, rather than being restricted to a wavelength optimally absorbed by a targeted chromophore. In fact, while the wavelengths selected normally have significant water absorption, it is desirable that the selected wavelengths is that they are not highly absorbed, even by water, so that the radiation can reach desired depths without losing substantial energy/photons to absorption. The concentration of photons/energy at treatment columns increases energy at these portions more than enough to compensate for reduced absorption at the wavelength utilized.

Any of a number of cosmetic conditions may be treated, improved and/or remedied in accordance with the methods and devices disclosed herein. For example, the disclosed devices and methods may be employed to treat scars, including acne scars, chicken pox scars, hypotropic scars and the like, for bumps in the skin resulting from scar tissue, for stretch marks, for treating certain parasites (e.g., intredermal parasites such as larva migrans), etc. The disclosed devices and methods may be employed for the removal of tattoos or pigmented lesions, particularly close to the skin surface, where other techniques frequently result in blistering and other skin problems. An improved technique which would permit the fading of such tattoos or pigmented lesions and/or the ultimate removal thereof in a gentle enough manner so as to not cause damage to the patient's skin or significant patient discomfort is also desirable. The devices and methods disclosed herein can be employed to treat vascular lesions including spider veins and other small veins. Where a vascular lesion at a selected depth is being treated, treatment parameters, including the electromagnetic energy source, the optical system, the targeted depth of treatment and the wavelength of the applied radiation are selected so that the at least one depth of the treatment portions are at the depth of the vessel being treated. Similarly, where the treatment is skin remodulation by treatment of collagen or hair removal, treatment parameters, including the electromagnetic energy source, the optical system, the targeted depth of treatment and the wavelength of the applied radiation are selected so that the at least one depth is the depth of interdermal collagen and the depth of at least one of the bulge and matrix of the hair follicle, respectively. The devices and methods disclosed herein may also be used to treat acne, to target and destroy pockets of fat, to treat cellulite and to treat other skin blemishes, and for treating various other conditions in the skin. The devices and methods disclosed herein can be used not only to remove wrinkles but also to remove other skin blemishes such as acne or chicken pox scars or other scars in the skin. Treatment with the devices and methods disclosed herein can increase the thickness-to-length ratio of the collagen in the area, thus increasing the collagen thickness, resulting in much of the improvement from skin rejuvenation/blemish removal being reasonably permanent. Other skin blemishes treatable by the teachings disclosed herein include stretch marks, which differ from wrinkles in that these marks are substantially flush with the surface, the collagen shrinkage and regeneration as a result of heating reducing these marks. Hypotropic scarring, the raised scars which occur after surgery or certain wounds, can also be treated by reducing blood flow to the vessels of the scar in much the same way port wine stains may be treated.

In addition to hair removal, treatment of vascular lesions, and skin resurfacing, the teachings of disclosed herein also be used to target and destroy a sebaceous gland or glands, for example to treat acne, to target and destroy pockets of subcutaneous fat, to treat cellulite and to do skin resurfacing on difficult areas, for example neck and hands, where the damage caused using standard skin resurfacing techniques does not normally heal. The treating of only small fractional regions of an overall treatment volume in such areas should leave sufficient undamaged skin structure for healing to occur. The methods and devices disclosed herein can be used to treat various skin cancers, PFB, and psoriasis.

### DETAILED DESCRIPTION OF THE FIGURES

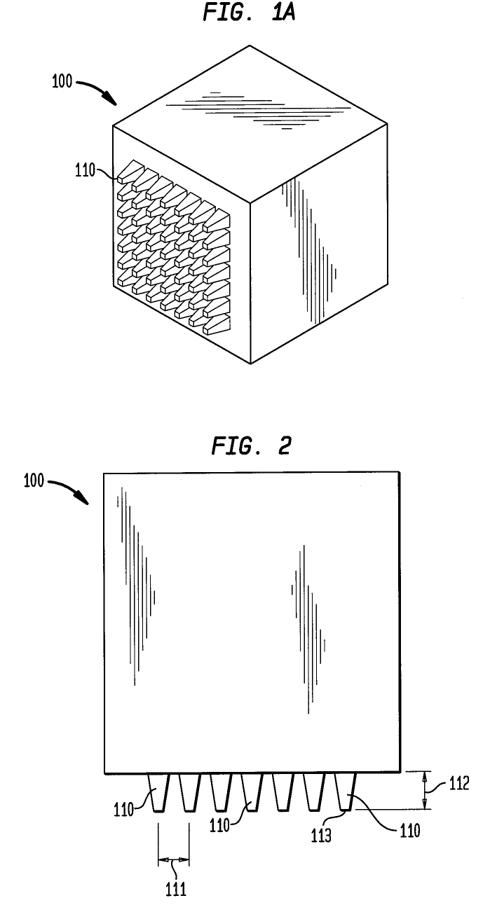

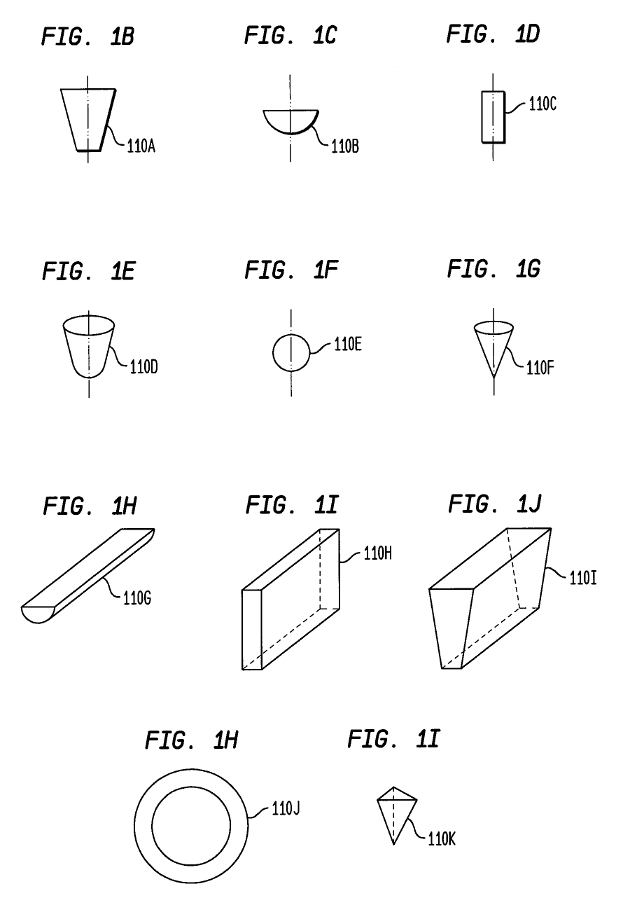

FIG. 1A is front perspective view of a deformation applicator.

FIG. 1B shows a point compression element having a trapezoid configuration.

FIG. 1C shows a point compression element having a half sphere configuration.

FIG. 1D shows a point compression element having a rectangle configuration.

FIG. 1E shows a point compression element having a parabola configuration.

FIG. 1F shows a point compression element having a ball configuration.

FIG. 1G shows a point compression element having a cone configuration.

FIG. 1H shows a point compression element having a cylindrical groove configuration.

FIG. 1I shows a point compression element having a rectangular groove configuration.

FIG. 1J shows a point compression element having a trapezoidal groove configuration.

FIG. 1K shows a point compression element having an annulus configuration.

FIG. 1L shows a point compression element having a pyramid configuration.

FIG. 2 shows a side view of the deformation applicator shown in FIG. 1A.

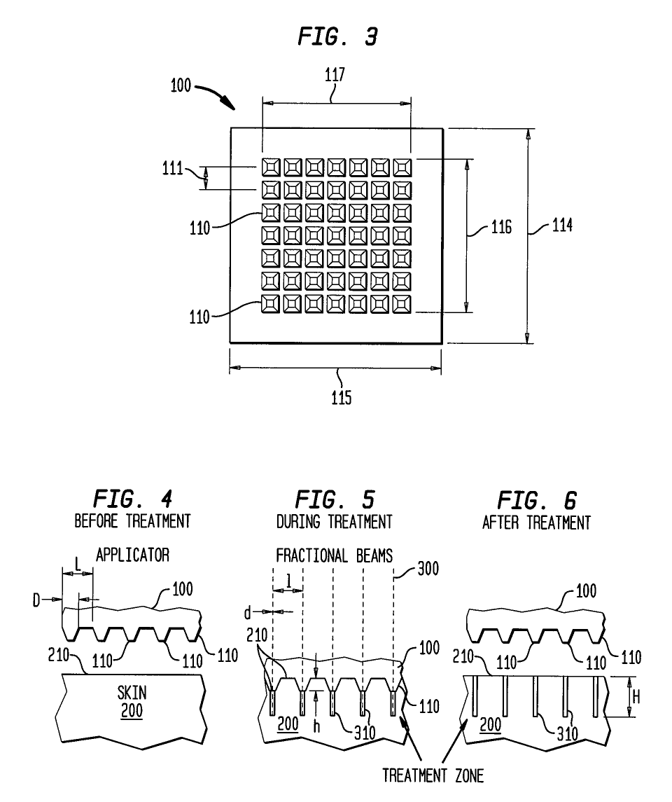

FIG. 3 shows a front Schematic View of the Deformation Applicator of FIG. 1A

FIG. 4 is a schematic view of treatment using the deformation application of FIG. 1A shown before treatment.

FIG. 5 is a schematic view of treatment using the deformation application of FIG. 1A shown during treatment.

FIG. 6 is a schematic view of treatment using the deformation application of FIG. 1A shown after treatment.

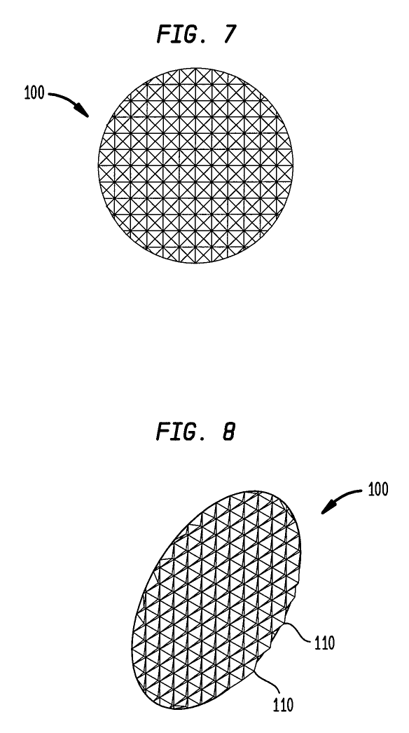

FIG. 7 is a front schematic view of another deformation applicator.

FIG. 8 is a front perspective view of the deformation applicator of FIG. 7.

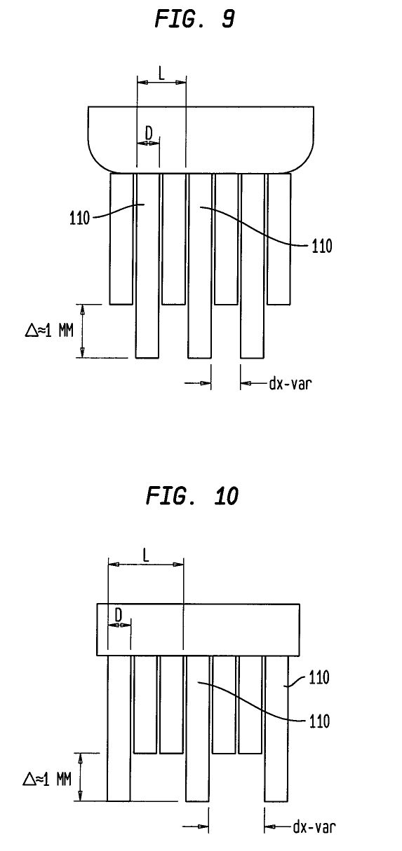

FIG. 9 is a schematic view of a deformation applicator in which the pitch is equal to two times the diameter of the point compression element(s).

FIG. 10 is a schematic view of a deformation applicator in which the pitch is equal to three times the diameter of the point compression element(s).

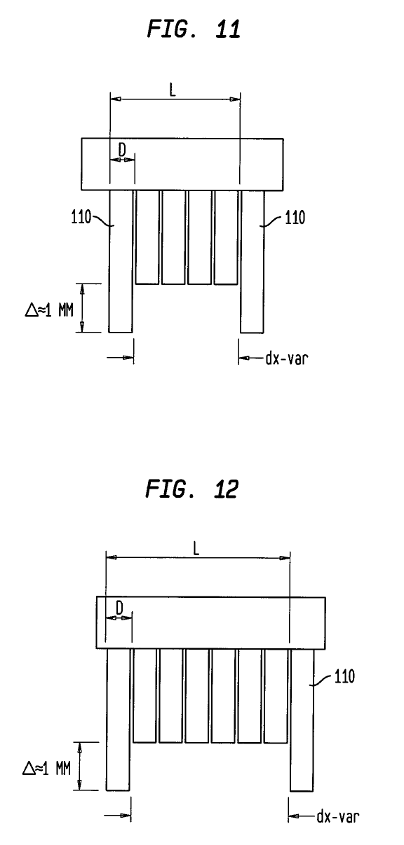

FIG. 11 is a schematic view of a deformation applicator in which the pitch is equal to five times the diameter of the point compression element(s).

FIG. 12 is a schematic view of a deformation applicator in which the pitch is equal to seven times the diameter of the point compression element(s).

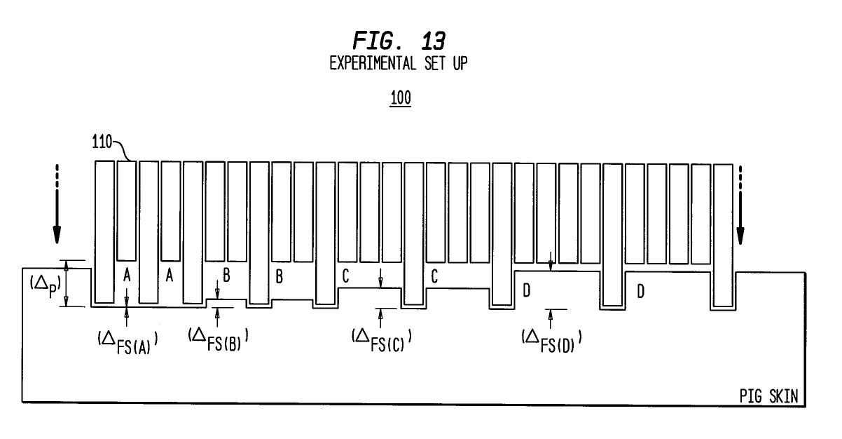

FIG. 13 is a schematic view of an experimental deformation applicator in which the pitch is varied across the length of the applicator and the deformation of tissue between adjacent point compression elements changes as the pitch is altered.

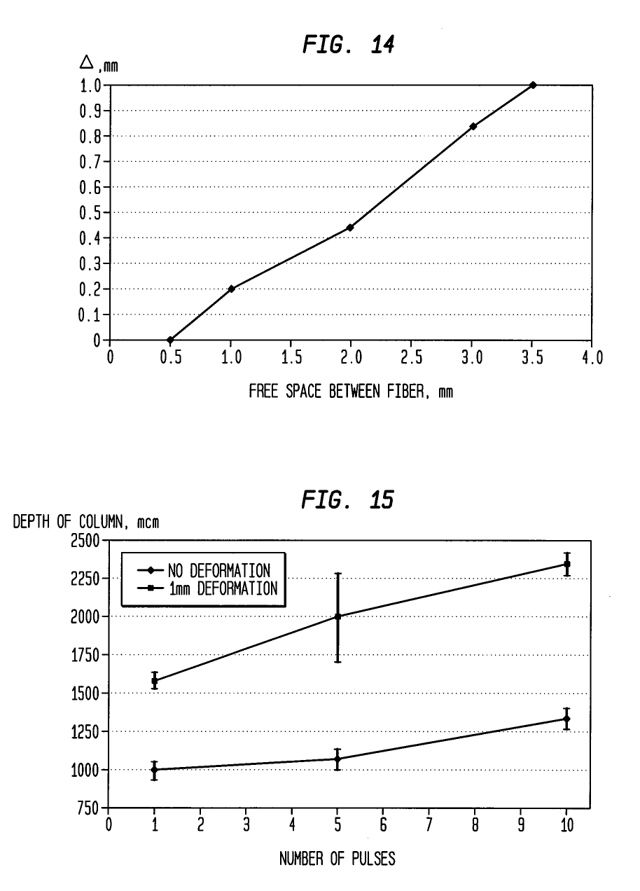

FIG. 14 is a graph showing the results of deformation testes with the Y-axis showing the measured deformation and the X-axis showing the free space between point compression element(s).

FIG. 15 is a graph comparing the depth of fractional treatment columns with an without 1 mm deformation of tissue and with or without stacking of pulses. The Y-axis shows the depth of the fractional treatment column formed in the tissue and the X-axis shows the number of pulses that were delivered.

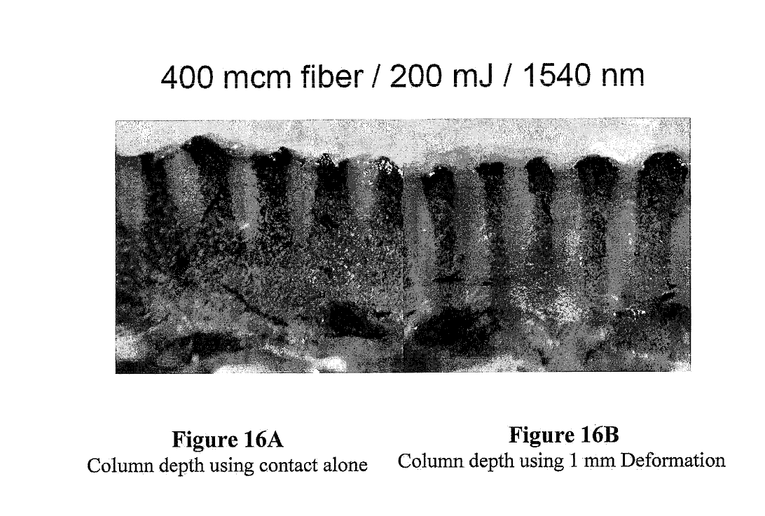

FIG. 16A shows an image of column of damage depth achieved using contact alone and without deformation.

FIG. 16B shows an image of column of damage depth achieved using deformation and with the same fiber diameter, mJ, and wavelength conditions as shown in the results of FIG. 16A.

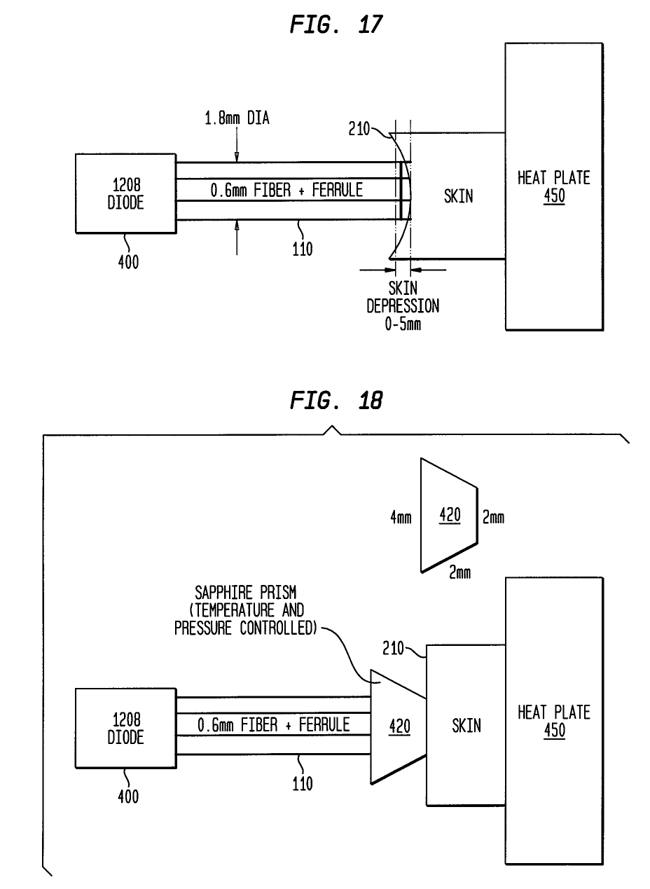

FIG. 17 shows the optical layout when a single fiber acts a point compression element.

FIG. 18 shows the optical layout when a single fiber together with an optical prism contacts the skin.

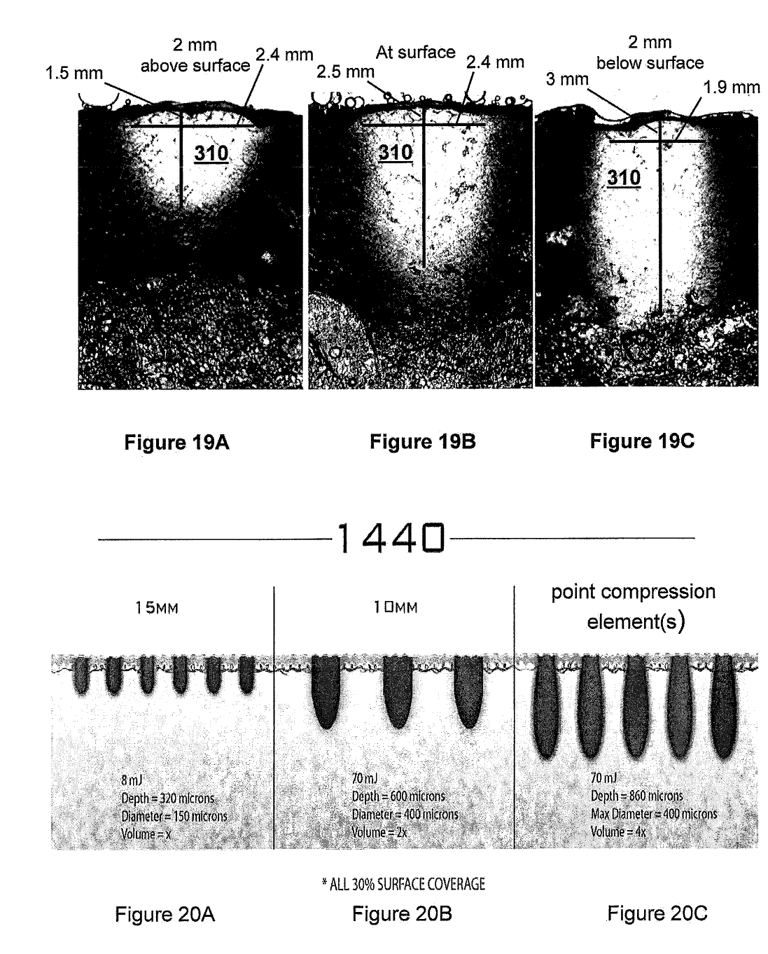

FIG. 19A is a parallel polarized image of pig skin tissue treated with the single fiber shown in FIG. 17 with the fiber 2 mm above the surface of the pig skin.

FIG. 19B is a parallel polarized image of pig skin tissue treated with the single fiber shown in FIG. 17 with the fiber at the surface of the pig skin.

FIG. 19C is a parallel polarized image of pig skin tissue treated with the single fiber shown in FIG. 17 with the fiber 2 mm below the surface of the pig skin.

FIG. 20A shows a computer simulation of skin tissue treated with a fixed surface coverage of 30% and with a 15 mm optic held at the surface of the skin.

FIG. 20B shows a computer simulation of skin tissue treated with a fixed surface coverage of 30% and with a 10 mm optic held at the surface of the skin.

FIG. 20C shows a computer simulation of skin tissue treated with a fixed surface coverage of 30% and with point compression element displacing at least a portion of the skin tissue.

### DETAILED DESCRIPTION

Disclosed herein is the discovery that by deforming skin or other tissue in relatively small areas, the electromagnetic radiation (“EMR”), particularly optical radiation such as visible and infrared light, can be delivered to the tissue more effectively. For example, a quantity “A” of EMR can be delivered more deeply into a deformed area of the tissue than if the same area of tissue were not deformed and the quantity “A” of EMR, e.g., the same amount of energy, were delivered. This phenomenon, referred to herein as deformation of tissue (e.g., micro-deformation of tissue), is believed to be particularly applicable to the fractional treatment of tissue with EMR, but also has application in other treatments as well, such as increasing the depth of penetration of non-fractional EMR treatments of tissue.

The electromagnetic device(s) disclosed herein can have a beam spot size that ranges from about 30 microns to about 3 mm, from about 50 microns to about 1 mm, or from about 50 microns to about 500 microns. The wavelength can range from about 290 nm to about 11000 nm. The pulse width can range from about 1 nanosecond to continuous wave, or from about 1 microsecond to about 100 milliseconds. The fluence can range from about 0.1 J/cm² to about 1000 J/cm². The depth of deformation into the skin (at the deepest point of deformation) can range from about 100 microns to about 3 mm as compared to the normal surface of the skin.

Also disclosed is the discovery that deformation of skin tissue (e.g., micro-deformation) provides local deformation using positive or negative pressure to enhance penetration of EMR during the fractional treatment of skin tissue. Without being bound to any single theory, it is believed that skin is compressible, but that tissue fluids (including water, lymph, and/or blood) are not. Since skin comprises 60-70% water, then the compressibility of skin arises in part from the displacement of tissue fluids including water. The deformed tissue accordingly has a modified tissue fluid (e.g., water) content that will change scattering and absorption. In addition to the changes in the optical properties of skin, the thermal properties also may change with compression. For example, with the displacement of tissue fluid (e.g., water), the average, local thermal properties such as thermal conductivity, specific heat, and thermal diffusivity will also change. Local deformation may also be referred to as point compression and/or tissue fluid displacement.

Significant deformation (e.g., micro-deformation) of skin can be achieved in relatively small area (the “deformation area” or “DA”) having a diameter (D). (Note that, although discussed in terms of a circular area having a diameter, many other shapes, configurations and dimensions of a deformed area may be employed. Further, the concepts can be applied also in terms of a small volume or “deformation volume” or DV). Skin may be deformed fractionally, i.e., with a distance between two deformed areas in which there is lesser or no deformation of tissue.

The amount of tissue deformation is significantly greater when accomplished over a number of relatively small areas than when accomplished over a single relatively larger area. The deformation (e.g., micro-deformation) can be performed using a one-dimensional or two-dimensional array or matrix of DAs. Although many configurations are possible, it is thought that the pitch (L) of a matrix of DAs be greater than the diameter (D) of the DAs, i.e., L/D>1. Even more preferably, the array or matrix of DAs has a ratio of L/D that is in the range of approximately 2 to 10. The treatment depth (H) can be increased significantly relative to the deformation depth (h) of the tissue. In other words, the resulting treatment depth (H) can be greater than and/or significantly greater than the deformation depth (h) of the tissue in some embodiments.

In one embodiment shown in FIGS. 1A, 2, 3, and 4-6, a deformation applicator 100 includes one or more point compression element(s) 110 (e.g., a set of protrusions extending from the face of the applicator). The point compression element(s) 110 can have any of a number of shapes and/or configurations. FIGS. 1B-1K show a variety of point compression element 110 configurations. Suitable point compression element(s) 110 can have several shapes and have symmetry about an axis and form a point compression, have symmetry about a plane and form a line compression, and have an annulus to produce a shape of treatment such as a circle, a square (not shown), a rectangle (not shown), a star (not shown), among others, for example. FIGS. 1B-1K and Table 1 disclose attributes of at least some of the possible point compression element(s) 110 that can be employed in accordance with this disclosure.

TABLE 1

| FIG. | Shape | Symmetry |
|---|---|---|
| 1B | Trapezoid 110A | About an Axis |
| 1C | Half Sphere 110B | About an Axis |
| 1D | Rectangle 110C | About an Axis |
| 1E | Parabola 110D | About an Axis |
| 1F | Ball 110E | About an Axis or About a Plane |
| 1G | Cone 110F | About an Axis |
| 1H | Cylindrical Groove 110G | About a Plane |
| 1I | Rectangular Groove 110H | About a Plane |
| 1J | Trapezoidal Groove 110I | About a Plane |
| 1K | Annulus 110J | About an Axis |
| 1L | Pyramid 110K | About an Axis |
| | Waveguide | Light propagates by total internal |
| | | reflection, this can be an optical fiber |

Referring now to FIGS. 1A, 2, 3, the applicator 100 has one or more point compression element(s) 110. Referring to FIG. 2, the point compression element(s) 110 have a length 112 that ranges from about 0.5 mm to about 10 mm, or from about 1 mm to about 6 mm, or from about 2 mm to about 4 mm. The length 112 is a measure of the staggered ends and/or protrusions 110 and their length is measured relative to the surface on which they are disposed which enables displacement and/or local deformation of tissue.

The distance 111 measured between the center of adjacent point compression element(s) 110 range from about 0.5 mm to about 10 mm, from about 1 mm to about 5 mm, or about 1.56 mm. The portion of the point compression element(s) 110 that contact the tissue surface and compresses the tissue (e.g., the contact compression surface 113) ranges from about 0.001 mm to about 10 cm, from about 0.2 mm to about 5 mm, from about 1 mm to about 2 mm, or about 0.54 mm. Depending upon the shape of a given protrusion of the point compression element 110, the contact compression surface 113 may contact a greater portion of the protrusion than is shown in FIG. 2. For example, in the point compression element 110B shown in FIG. 1C the entire curved portion of the half sphere contacts the tissue. Now referring to FIG. 3, in one embodiment, the face of the applicator 110 has an applicator length 114 that ranges from about 2 mm to about 20 cm, from about 5 mm to about 1 cm, from about 10 mm to about 60 mm, or from about 15 mm and has an applicator width 115 that ranges from about 2 mm to about 20 cm, from about 5 mm to about 1 cm, from about 10 mm to about 60 mm, or from about 15 mm. In one embodiment, the face of the applicator 110 has a point compression element array length that ranges from about 0.5 mm to about 18 cm, from about 3 mm to about 1 cm, from about 8 mm to about 50 mm, or from about 10.42 mm and has a point compression element array width that ranges from about 0.5 mm to about 18 cm, from about 3 mm to about 1 cm, from about 8 mm to about 50 mm, or from about 10.40 mm.

Referring to FIGS. 4-6, the point compression elements 110 are configured to contact the tissue 200 skin 210 during an EMR treatment. In one embodiment, all or a portion of the applicator 100 is made from an optically transmissive substance, such as sapphire. In another embodiment, all or a portion of the point compression element(s) 110 are made from an optically transmissive substance, such as sapphire. In one embodiment, point compression element(s) 100 are at least partially transparent to enable EMR (e.g., optical radiation) to travel therethrough. In one embodiment, one or more point compression element(s) are have a hole and/or plug made of transparent material such as sapphire that enables EMR to travel through the point compression element(s), optionally, portions that surround by transparent material are non transparent (e.g., made from metal(s) or polymers(s), etc.)

Referring now to FIGS. 4-6, the point compression elements 110 (e.g., the protrusions for an array of deformation applicators with size (D) and pitch (L)) can be coextensive with and/or correspond to a matrix of fractional beams. FIG. 5 shows fractional beams 300 that are coextensive with the point compression elements 110. In one embodiment, a matrix of fractional beams 300 each has a diameter (d) that is less than (D) and a pitch (l) that is equal to (L). In one embodiment, the applicator is configured to be attached to a suitable EMR treatment device such as a fractional treatment device or other device.

Referring to FIGS. 4-6, the deformation applicator 100 is positioned over the skin surface 210 of a region of tissue 200. In FIG. 5, one or more point compression element(s) 110 are pressed into the skin surface 210 (e.g., the epidermis). A portion of the skin surface 210 is compressed and/or deformed under the point compression element 110. In the space between adjacent point compression element(s) 110 the skin surface 210 is not compressed and/or is not compressed in the manner that the skin surface 210 is compressed under the point compression element(s) 110. At least some tissue fluid (e.g., water, blood, lymph etc.) is displaced by the point compression element(s) 110. In one embodiment, the amount of tissue fluid displacement varies as a function of the length of time that the applicator 100 and its point compression element(s) 110 are pressed onto the surface of the skin 210. The longer the point compression element(s) are pressed onto the surface of the skin 210 prior to treatment with EMR the greater the opportunity for tissue fluid displacement. In one embodiment, the point compression element(s) are pressed onto the surface of the skin 210 for a time that ranges from about 0.5 seconds to about 10 minutes, from about 1 second to about 1 minute, from about 2 seconds to about 10 seconds, or for a length of time determined suited to the particular desired outcome by the practitioner performing the procedure. The external skin surface 210 may retain the imprint of the point compression element(s) 110 for a period of time after the they are pressed onto the surface of the skin 210 until, for example, the tissue fluid returns to the regions of deformation to reduce and/or remove the appearance of the imprint on the skin surface 210.

Fractional beams 300 are delivered into the skin surface 210 and into the tissue 200 such that one fractional beam travels through a single point compression element 110 and into the compressed tissue in contact with the point compression element 110. Treatment zone(s) of damaged and/or treated tissue 310 are created in the tissue 200. The zones of treated tissue 310 are deeper than would have been achieved if the same fractional beams 300 (e.g., the same EMR) were provided in the absence of compression of the tissue 200 with the point compression element(s) 110. More specifically, tissue fluid displacement occurs where the point compression element(s) are pressed onto the surface of the skin 210. There may be some tissue fluid displacement in the regions of tissue 200 between adjacent point compression element(s) 110, however, the amount of tissue fluid displacement in the tissue 200 compressed by the point compression element(s) 110 is greater than and/or significantly greater than any displacement in the regions of tissue 200 between adjacent point compression element(s) 110.

These changes in compressed tissue may combine to enhance desired thermal effects arising from photothermolysis. For example, the adiabatic application of radiation to heat tissue to a desired temperature (e.g., for coagulation) will become possible at lower laser power levels where there is a decrease in thermal diffusivity of the tissue (with all other parameters the same). The adiabatic application of radiation means that the heat is delivered to the tissue at a faster rate than the rate at which the object (e.g., the treated tissue) will cool. In particular, the adiabatic condition is characterized by the thermal relaxation time, τ_{R}, which is inversely proportional to the thermal diffusivity

$${τ}_{R} ∝ \frac{{D}^{2}}{α} \qquad ( Formula 1 )$$

Here D is a length characteristic of the geometry of the EMR beam and α is the thermal diffusivity that depends on the following thermal properties of the tissue:

$$α ∝ \frac{κ}{ρ {C}_{p}} \qquad ( Formula 2 )$$

κ is the thermal conductivity of the tissue, ρ is the density of the tissue and Cₚ is the specific heat of the tissue. If the tissue thermal diffusivity decreases as water concentration decreases, then the relaxation time increases and the achievable temperature rise also increases for a given incident EMR power. As a result, where a decrease in diffusivity results from a decrease in tissue fluid (e.g., water) concentration due to deformation (e.g., micro-deformation) and/or point compression, one can reduce the input power required to achieve a desired temperature rise. It is believed that the level of deformation (e.g., micro-deformation) and/or point compression can be optimized to achieve the desired diffusivity properties. The time of tissue deformation and/or point compression prior to applying EMR can be selected and/or altered to allow tissue fluid (e.g., water and/or collagen) concentration to decrease. Generally, applying a point compression element for a longer period of time enables a greater quantity of tissue fluid to displace thereby further reducing tissue diffusivity and providing the benefits associated therewith.

The thermal capacity of the tissue is altered (e.g., decreased) by employing local deformation, also referred to as point compression, which results in tissue fluid displacement. This allows for enhanced cooling of the deeper tissues, because there is better thermal contact between the point compression element(s) and the surface of the subject's skin (e.g., the stratum corneum of the subject's skin). In addition, cooling of deeper tissues is also enhanced by decreasing the tissue fluid content, because employing one or more point compression element(s) on the surface of a tissue lowers the heat capacity of the tissue in contact with the point compression element, e.g., the portion of the skin surface in contact with a point compression element having a pin-like shape (see, e.g., FIGS. 1B and 1G). In addition to decreased absorption of the light by skin (e.g., the water in skin tissue) the reduction in heat capacity enhances cooling to better preserve the D/E junction. Further, all or a portion of the deformation element may be cooled. In one embodiment, only the point compression element(s) are cooled. In another embodiment, only a portion of the point compression element(s) are cooled (e.g., only the distal end of the point compression element(s) in contact with the skin surface are cooled). For example, the point compression element optic may be cooled by any of a number of means (e.g., gas, liquid (water, refrigerant, or any coolant), thermoelectric, vaporization, and/or melting). Optionally, the point compression element is actively cooled and a regulated flow of coolant is provided to the point compression element optic to ensure a substantially consistent temperature. Employing cooling enables higher energy to be delivered to the skin tissue by controlling and/or limiting damage to the surface of the skin tissue (i.e., down to and including the dermal-epidermal junction (i.e., the D/E junction). In this way adverse events such as hyper-pigmentation and/or hypo-pigmentation may be minimized and/or avoided.

Also, a decrease in tissue fluid (e.g., water) content caused by tissue compression and/or deformation (e.g., micro-deformation) may cause a decrease in the tissue's scattering and absorption properties such that light can penetrate more deeply into the tissue compared to a treatment using the same energy and other parameters but lacking deformation (e.g., micro-deformation) of the tissue with the point compression element. In cases where the tissue is compressed, the deformed tissue has a smaller volume and, following mechanical relaxation, the treated tissue extends to a larger volume. In cases where the tissue is extended, the opposite is true, i.e., the deformed tissue has a larger volume and, following mechanical relaxation, the treated tissue extends to a smaller volume.

In addition to allowing for deeper penetration of light and deepening of the treatment zone, by employing a point compression element the area of “damage” or “treatment” induced by the EMR may be reduced at the superficial layers of the skin, e.g., at the epidermis. Superficial skin layer damage may be reduced and/or avoided because better contact with the skin provides better heat transfer from the skin and/or the subcutaneous tissue (e.g., fat tissue) to the contacting optic. Superficial skin layer damage may be reduced and/or avoided because a decrease in local tissue fluid concentration and the resulting decrease in tissue scattering will lower the energy deposited in the upper layer to decrease the amount of thermal damage. Thus employing a point compression element and to achieve local deformation enables one to increase the volume of tissue treated without increasing damage at the dermal-epidermal junction. Reducing the damage at the dermal-epidermal junction can decrease the risk of, for example, hypo-pigmentation and hyper-pigmentation. In addition, employing a point compression element to achieve local deformation enables the a tissue volume to be treated with a relatively lower power device than would be required in the absence of location deformation provided by, for example, a point compression element.

Referring again to FIGS. 4-6, during treatment, the applicator 100 is pressed into the surface 210 of an area of tissue 200, in this case skin. The one or more point compression element(s) 110 (e.g., protrusions) on the applicator 200 press onto the surface 210 of the skin and, in one embodiment, do not pierce and/or cut into the skin surface 210. The tissue is thereby deformed by the one or more point compression element(s) 110 pressed into the skin surface 210. While deformed, an array of beams 300 of EMR, each having a diameter (d) and a pitch (l), is applied to the tissue. The array of EMR beams 300 creates an array of treatment zones 310 each having a treatment depth (h). The applicator 100 is then removed from the tissue 200, and the tissue relaxes resulting in a treatment depth (H) for each treatment zone 310. In one embodiment, the diameter (d) of each beam is less than the size (D) of the point compression element (e.g., the protrusion) through which the beam 300 is applied to the surface 210 of the tissue 200. Another embodiment of the applicator 100 is shown in FIG. 7 through 8. The applicator 100 shown in FIG. 7 is similar to the applicator in FIG. 1, but has a circular configuration, which is different from the square configuration shown in FIG. 1. The face of the applicator 100 that contacts the skin surface during use for treatment of tissue is circular and each point compression element 110 (e.g., each protrusion on the applicator 100), which collectively form the array of DAs, have a pointed pyramid-like shape similar to the shape depicted in FIG. 1K.

Many other embodiments of the device and method discussed in connection with FIGS. 1A-1L are possible. For example, the tissue (e.g., the skin) can be expanded rather than compressed. The point compression elements (e.g., the protrusions) can be shaped and sized differently, including, without limitation, longer, shorter, narrower, wider, rounded, pointed, squared cross sectional areas, and the cross sectional areas and shapes disclosed in connection with FIGS. 1A-1L, for example. In one embodiment, a single applicator can have a point compression elements having varying sizes (e.g., length, width) and varying shapes (e.g., a pyramid and a groove). The point compression elements (e.g., protrusions) can be elongated lines or ridges, pin-points, circular, oval, rectangular, or square in shape, or can have many other shapes. Further, a single array can include regularly spaced and uniformly shaped protrusions or the dimensions of the array and/or the protrusions in the array can be varied to many other configurations. Additional forms of deformation or a combination of forms of deformation can be used. For example, vacuum pressure applied at the perimeter of an applicator (e.g., in the region of the applicator length 114 and the applicator width 115 as depicted in FIG. 3) can be used to stretch the tissue over the applicator as the skin is deformed with the protrusions of the applicator. In various embodiments, different fractional treatment devices can be used, for example, a device having an array of micro-lenses can be used (e.g., can be used in a “stamping mode”) as well as a device that includes a scanner, e.g., that can be used in a scanning mode. Additionally, other embodiments can be employed with non-fractional EMR-based devices, as well as devices (fractional and non-fractional) that use other types of energy, including, without limitation, optical, ultrasound, microwave, and RF energy sources. The applicator can be formed as a permanent attachment incorporated into a treatment device or can be detachable, e.g., as part of a set of applicators for various purposes and/or as a disposable applicator.

### Experimental Tests of Various Micro-Deformation Applicators

Devices similar to the embodiment described in conjunction with FIG. 1A were tested in an in vitro study. The applicator embodiments used and the results of the study are described in conjunction with the following figures. As shown in the schematic diagrams of FIGS. 9-12 a set of 500 micrometer fibers were used. FIG. 9 shows a set of 500 micrometer fibers that each have a circular cross section in which the pitch (L) is two times the diameter (D) of the fibers. The point compression element(s) 110 are staggered with ends displaced approximately 1 mm from the ends of adjacent fibers to provide local deformation of tissue.

FIG. 10 shows a set of 500 micrometer fibers that each have a circular cross section in which the pitch (L) is three times the diameter (D) of the fibers. The point compression element(s) 110 are staggered with ends displaced approximately 1 mm from the ends of adjacent fibers to provide local deformation of tissue.

FIG. 11 shows a set of 500 micrometer fibers that each have a circular cross section in which the pitch (L) is five times the diameter (D) of the fibers. The point compression element(s) 110 are staggered with ends displaced approximately 1 mm from the ends of adjacent fibers to provide local deformation of tissue.

FIG. 12 shows a set of 500 micrometer fibers that each have a circular cross section in which the pitch (L) is seven times the diameter (D) of the fibers. The point compression element(s) 110 are staggered with ends displaced approximately 1 mm from the ends of adjacent fibers to provide local deformation of tissue.

A test employing the applicators shown in the schematic diagrams of FIGS. 9-12 evaluated the effect of varying the distance between the fibers configured to deform tissue (see, e.g., dx-var on FIGS. 9-12), this spaces is also referred to as the free space (see, e.g., FIG. 14).

Referring to FIGS. 9-13, the distances between the extended fibers (e.g., the point compression element(s) 110) were varied to examine the effect of the various dimensions on the tissue (i.e., pig skin). As shown in FIG. 13, the deformation of tissue between adjacent extended fibers increased as the pitch of the extended fibers increased (i.e., as the distance between the fibers configured to deform tissue is increased and as the free space between adjacent point compression elements is increased). The applicator 100 is compressed into the pig skin. Generally, a relatively small amount of deformation (A) occurred when the pitch (L) was approximately equal to the diameter (D) of the fibers, while the tissue deformation increased as the pitch (L) increased. More specifically, referring to FIG. 14, where the free space A was approximately equal to the diameter of the point compression element 110, the deformation in the free space A (Δ_{FS(A)}) is very small.

Where the free space B was approximately equal to two times the diameter of the point compression element 110, the deformation in the free space B (Δ_{FS(B)}) is still small, but is larger than the deformation in the free space A (Δ_{FS(A)}). Where the free space C was approximately equal to three times the diameter of the point compression element 110, the deformation in the free space C (Δ_{FS(C)}) is larger than the deformation in the free space B (Δ_{FS(B)}) and is larger than the deformation in the free space A (Δ_{FS(A)}). Where the free space D was approximately equal to four times the diameter of the point compression element 110, the deformation in the free space D (Δ_{FS(D)}) is larger than the deformation in the free space C (Δ_{FS(C)}), is larger than the deformation in the free space B (Δ_{FS(B)}), and is larger than the deformation in the free space A (Δ_{FS(A)}). There the deformation in the free space is relatively larger e.g., the deformation of the free space D (Δ_{FS(D)}) pig skin tissue contacts the contact compression surface of the point compression element(s) 110 that surround the free space D, but in addition at least a portion of the tissue between adjacent point compression element(s) 110 contact the applicator (e.g., shorter fibers between the adjacent point compression elements) in the region of the free space D. In addition, referring still to FIG. 13, the region of greatest deformation by the point compression element(s) 110 is where the length of the point compression element 110 (i.e., Δ_{P}) is substantially the same as the deformation of the free space D (Δ_{FS(D)}).

Referring to the graph in FIG. 14, the results of deformation testing of pig skin described in relation to FIGS. 9-13 are shown. The Y-axis shows the measured deformation (Δ, m) of the tissue in the direction of the depth of the skin tissue (approximately normal to the skin surface). The X-axis marked free space between fiber has the following relationship with the pitch (L), at 0.5 mm, L=2D; at 1.0 mm, L=3D; at 2.0 mm, L=5D; at 3.0 mm, L=7D; and at 3.5 mm, L=8D. The measured deformation increased as the free space between the adjacent point compression elements (e.g., protrusions and/or extended fibers) increased. The graph at FIG. 14 shows that the increase in the measured deformation of the tissue has a substantially linear relationship with the increase in free space between adjacent point compression elements (e.g., protrusions and/or extended fibers).

Following the deformation tests, tests were conducted to compare the results of fractional columns created by irradiating pig skin tissue using a set of fibers placed in contact with the surface of the pig skin tissue (and without deformation) and fractional columns created using the same device, but with the device further pressed into the tissue to create various magnitudes of deformation (e.g., micro-deformation) using point compression elements in a direction roughly normal to the upper surface of the pig skin tissue A summary of the results of the tests are shown in the graph shown in FIG. 15. In FIG. 15 the X-axis shows the number of pulses that were delivered and the Y-axis shows the depth of the fractional treatment column formed in the tissue as a result of the EMR delivered via the varying pulses. During each of the tests summarized in FIG. 15, the applied wavelength was 1540 nm, using 200 mJ, at a fluence of 125 j/cm², at a pulse width of 13.5 ms and at 0.2 Hz the number of pulses varied during the testing such that one test included N=1 pulse, another test included N=5 pulses, and another test included N=10 pulses. FIG. 15 shows that employing point compression element(s) at a deformation depth of 1 mm in tissue together with fractional EMR directed through each point compression element when located at 1 mm depth in tissue enables formation of a column deeper in tissue than is possible with fractional EMR in the absence of deformation. In addition, use of 1 mm of deformation enables column formation in a single pulse at a depth that is deeper than is available with ten “stacked” pulses (e.g., ten pulses of fractional EMR in the same location in a single islet of damage) in the absence of and with no deformation.

FIG. 16A shows an image of column depth achieved using contact alone (without deformation) and applying 1540 nm at 200 mJ via a 400 μm fiber. FIG. 16B shows an image of column depth achieved using 1 mm deformation and applying 1540 nm at 200 mJ via a 400 μm fiber. Comparing applying deformation (see, FIG. 16B) enables formation of column depths that are much deeper than with contact alone and in the absence of deformation (see, FIG. 16A).

Deformation applicators (e.g., micro deformation applicators including one or more point compression element(s)) were also tested on ex vivo porcine skin and in human skin. FIGS. 17-18 depict the optical layouts where a single pin in the form of a 0.6 mm diameter fiber acts as a point compression element. The 0.6 mm diameter fiber is disposed in a ferrule, a tube that surrounds all or a portion of the 0.6 mm diameter fiber, the outsider diameter of the ferrule enclosing the 0.6 mm fiber is 1.8 mm. Laser emission from a diode bar 400 at 1208 nm is coupled into a 600 micron diameter fiber, which acts as a point compression element 110. FIG. 17 shows a system where the 600 micron fiber 110 is applied directly to the skin surface 210 and compression is applied by the 600 micron fiber, which acts as a point compression element 110 to deform the skin 210 by a specified amount that ranges from 0 mm to 5 mm. FIG. 18 shows a system where the output light from the 600 micron fiber is applied to the skin surface 210 through an intermediate window 420, a sapphire pyramid optic. This sapphire pyramid optic 420 is then applied to the skin surface 210 and the skin is compressed below the pyramid optic 420. The portion of the pyramid optic 420 that contacts the skin surface 210 measures 2 mm and the end of the bare fiber that acts as the optical compression element 110 is about 2 mm from the surface of the skin 210. The setup shown in FIG. 18 may be used with or without a lens for focusing the micro beam.

The results demonstrate an increase in the depth of the coagulation zones with compression. FIGS. 19A-19C provides parallel-polarized images of pig skin tissue treated with the single bare fiber in FIG. 17, the treated tissue and/or damaged tissue 310 becomes quasi transparent. The single bare fiber shown in FIG. 17 has no adjacent fibers to share the tensile load of the skin. FIGS. 19A-19C show the parallel-polarized images of 150 micron thick pig skin after treatment with 2.5 Watts of laser radiation for 10 seconds. FIG. 19A shows the results where the fiber is 2 mm above the surface of the pig skin these results are achieved with the device of FIG. 17 is positioned 2 mm above the surface of the pig skin. FIG. 19B shows the results where the fiber is at the surface of the pig skin, these results are achieved with the device of FIG. 17 where the end of the point compression element is placed at the surface of the pig skin, but is not pressed into to deform the pig skin. FIG. 19 C shows the results where the fiber is pushed about 2 mm below the surface of the pig skin; these results are achieved with the device of FIG. 17 where the end of the point compression element 110 is pushed into the surface of the pig skin to deform the pig skin. The results in FIGS. 19A-19C demonstrate an increased depth of coagulation with compression. Increased compression caused by deforming the skin to 2 mm (FIG. 19C) increases coagulation depths by 20% as compared to the results shown in FIG. 19B where the fiber was not compressed into the skin, but was at the surface of the skin. Generally, the deeper the pin is pressed into the skin surface, the deeper the depth of damage into the tissue.

In addition, referring still to FIGS. 19A-19C, the diameter of damage at the surface of the compressed skin is less than the damage at the surface of the skin where the skin is not compressed. For example, the compressed tissue damage at the skin surface is 22% narrower in diameter than the non compressed skin. Avoiding and/or lessening damage at the skin surface can lessen the likelihood of adverse events such as hypo-pigmentation and/or hyper-pigmentation. Further, in some embodiments, dermal-epidermal separation can be reduced by employing local deformation (e.g., point compression element(s) to the skin surface).

In one embodiment, a fractional treatment at a wavelength of 1440 nm at 70 mJ was performed with and without location deformation employing one or more point compression element(s). As a result of the local deformation, the dermal-epidermal separation was reduced by from about 15% to about 30% and the depth was increased from about 20% to about 50% relative to the fractional treatment performed without local deformation, e.g., without compression applied by point compression element(s) to the skin surface. Avoiding and/or lessening the dermal-epidermal damage is important to avoid and/or lessen adverse events including hyper-pigmentation and hypo-pigmentation.

In one embodiment, employing the devices and methods to deform tissue using the one or more point compression element(s) enables the permeability of the stratum cornea and/or the epidermis to be controlled. More specifically, the deformation of the tissue achieved using the one or more optical compression element(s) stretches the stratum cornea in a manner that is remarkable and/or dramatic as compared with deformation provided using an apparatus that covers a larger area than the point compression elements such that there is not much deformation in the free space between point compression element(s).

In one embodiment, a compound is delivered to the surface of the skin (e.g., the epidermis) prior to treatment with the device(s) and/or the method(s) employing one or more point compression element(s) disclosed herein. Alternatively or in addition, suitable compounds can be delivered through the stratum cornea, into the stratum cornea, through the epidermis, into the epidermis, through the dermis, or into the dermis in accordance with the device(s) and/or the method(s) employing one or more point compression element(s) disclosed herein. Suitable compounds can enhance the benefits available from light treatment (e.g., a collagen promotion compound such as vitamin A or vitamin C may be employed). Others suitable compounds can enhance healing from light treatment, for example, anti inflammatory compounds may be employed to potentially decrease the side effects of treatment (e.g., antihistamine to decrease erythema). Other suitable compounds can have a refractive index that is close to or matches the refractive index of the area that is targeted by the light (e.g., interstitial fluids in the tissue, collagen, and other proteins).

In another embodiment, chromophores such as dyes, organic dyes, or carbon particles are delivered through the stratum cornea, into the stratum cornea, through the epidermis, into the epidermis, through the dermis, or into the dermis in accordance with the device(s) and/or method(s) employing one or more point compression element(s) disclosed herein. In one embodiment, after delivery of the chromophore to the desired location, light (e.g., light targeting the chromophores) is selected to destroy the tissue inch which the chromopores are resident (e.g., the stratum cornea, the epidermis, and/or the dermis). In one embodiment, light treatment of the chromophores cause a sufficient localized temperature rise to alter the permeability characteristic of skin (e.g., SC) can be effected.

Referring now to FIGS. 20A-20C, it is possible to increase the volume of treatment while maintaining a fixed coverage surface. In this way, the risk of adverse events associated with excess treatment of the dermal-epidermal junction can be lessened and/or avoided while achieving a greater treatment volume. FIGS. 20A-20C shows a computer simulation of skin tissue treated with a fixed surface coverage of 30%, such that in a given region of skin only 30% of the surface of the skin is treated. The computer simulation depicted in FIGS. 20A-20C is in good agreement with treatment column profiles (e.g., of coagulated columns) seen experimentally.

FIG. 20A shows a 15 mm optic provided at a wavelength of 1440 nm and at 8 mJ, which achieves a depth of 320 microns and a maximum diameter of 150 microns, the distance between adjacent 15 mm optics is about 0.6 mm with coverage of 30% at the surface of the skin tissue. FIG. 20B shows a 10 mm optic provided at a wavelength of 1440 nm and at 70 mJ, which achieves a depth of 600 microns and a maximum diameter of 400 microns the distance between adjacent 10 mm optics is about 1 mm with coverage of 30% at the surface of the skin tissue. Finally, FIG. 20C shows one or more point compression array element(s) provided at a wavelength of 1440 nm and at 70 mJ, which achieves a depth of 860 microns and has a maximum diameter of 400 microns with coverage of 30% at the surface of the skin tissue. The diameter of each fraction of treated tissue at the skin surface is less in FIG. 20C than in FIG. 20B, despite that both treatment models use a 1440 wavelength at 70 mJ. This difference appears to be due to use of the point compression element(s) applying pressure to the tissue and the resulting tissue fluid displacement, which lessens damage at the dermal-epidermal compared with a treatment at the surface of the skin and in the absence of use of point compression element(s). Because the damage at the dermal-epidermal junction is lessened, the related risks of hyper-pigmentation and hypo-pigmentation associated with excess damage, e.g., from about 30% to about 95% damage, of from about 35% to about 65%, or about 50% damage at the surface of the skin, is also reduced. The volume of treatment in the point compression element model to displace tissue and depicted in FIG. 20C is two times the volume of treatment in the model depicted in FIG. 20B that is at the surface of the skin tissue and does not apply displace the tissue. Further, other than the use of displacement by the point compression element(s) and treatment at the surface of the skin, the models at FIGS. 20B and 20C use all of the same conditions (e.g., 1440 nm, 70 mJ). Finally, the volume of treatment in the point compression element model to displace tissue and depicted in FIG. 20C is four times the volume of treatment in the model depicted in FIG. 20C that is at the surface of the skin tissue and does not apply displace the tissue. The cross sectional profile of the treated tissue modeled in FIG. 20C can be described as a teardrop, whereas the cross sectional profile of the treated tissue modeled in FIGS. 20A and 20C can be described as a column.

In some embodiments, a deformation applicator including point compression element(s) is employed together with stacking the pulses to thereby enhance penetration of the tissue treatment region. Where a stacked pulse strategy is employed, the point compression element(s) are compressed into the tissue surface and without moving the applicator EMR is applied to the tissue surface repetitively at some fixed interval. When using stacked pulses, the EMR can be applied by, for example, firing a laser, repetitively in a single treatment zone or within a single array of treatment zones without moving the applicator. EMR can be applied from about 2 times to about 20 times or from about 2 times to about 5 times. Where point compression element(s) are employed with a stacked pulse strategy the impact of maintaining the point compression element in a single location without movement can be to enhance displacement of tissue fluid to thereby improve scattering and absorption and improve the depth of coagulation due to the stacked pulses.

### Combination Fractional Ablative and Fractional Non-Ablative

Fractional non-ablative techniques result in coagulation of tissue and other forms of thermal injury, e.g. cell death, cell apoptosis and/or heat shock and/or protein generation. Coagulation and related thermal injury are important factors in skin rejuvenation, but they are limiting factors for maximizing single treatment coverage of fractional non-ablative deep treatments. More specifically, the amount of coverage in a fractional non-ablative treatment is limited by the risk of long term side effects (e.g., hyper pigmentation). Coverage may refer to the area of damage (relative to the total treatment area). More specifically, damage is created in a volume of tissue and the shape of this volume can be a column. This column of damage may assume the shape of e.g., a cylinder, tear drop, arrow, dagger or wedge or any other geometric shape with symmetry. Slicing a plane through these volumes of treated tissue whereby the plane is parallel to the skin surface enables quantification of the coverage in that plane. The coverage in this plane is defined as the total area of damage divided by the total area. Coverage at planes of different depths can vary. For example, if the column of damage has a cylindrical shape, the coverage is substantially the same at different depths. If the column of damage is of a tear drop shape, then the coverage will increase with depth from the skin surface, go through a maximum and then decrease with increasing depth. For example, where non-ablative treatment such as Lux1540 are employed, the collagen denaturation profile may be non-cylindrical and therefore the coverage at a given depth below the dermal-epidermal junction (the D/E junction can) be greater and/or less than the coverage at the D/E junction. In these instances, one can define coverage either to be the coverage at the D/E junction or the coverage at the depth (e.g, one of several depths in the tear drop shape damage profile) causing maximum coverage (i.e., that has the largest area at a given depth).

Generally non-ablative fractional treatment involves a treatment coverage area that is close to but does not reach the quantity of coverage that is known to cause long term side effects (e.g., hyper pigmentation) this is referred to as the maximum coverage for the fractional non-ablative treatment modality. As a result, multiple non-ablative treatments (e.g., three to four treatments) each separated by healing time are generally required to treat a condition (e.g., dyschromia). Generally, the maximum coverage for a single fractional non-ablative treatment is about 30% (e.g., treats the fraction of the tissue exposed to the device and the fraction is 30%), or from about 20% to about 40%.

Likewise, the amount of coverage in a fractional ablative treatment is limited by the risk of long term side effects (e.g., hyper pigmentation) and the risk of extensive downtime. Generally, ablative fractional treatment involves a single treatment. Generally, the maximum coverage for a single fractional ablative treatment modality is from about 30% to about 40%. Depending on the depth of treatment and the percentage coverage employed (the coverage ranges from about 10% to about 40%) the amount of downtime associated with a fractional ablative procedure ranges from about 2 days to 7 days.

The non-ablative treatments include a wavelength range of from about 900 nm to about 2600 nm. The ablative treatments include a wavelength range of form about 2600 to about 11000 nm. In one embodiment, the non-ablative treatment occurs prior to the ablative treatment. In another embodiment, the ablative treatment is conducted prior to the non-ablative treatment. In accordance with the methods disclosed herein, two separate electromagnetic radiation devices, one non-ablative and the other ablative, are employed to conduct the combination non-ablative and ablative treatment in a single session. In another embodiment, a single electromagnetic radiation device features two sources, one ablative and the other non ablative.

In still another embodiment, a single electromagnetic radiation device emits a wavelength range in the non-ablative range, i.e., from about 900 nm to about 2600 nm. The laser is enhanced by employing a coherent pump laser that coverts the non ablative electromagnetic radiation device to make it ablative such that is emits a wavelength range in the ablative range, i.e., from about 2600 nm to about 11000 nm. A coherent pump laser is simply one exemplary converter that can convert a non-ablative electromagnetic radiation device to operate as an ablative device; any other suitable converters are contemplated herein and may be employed. The electromagnetic device disclosed herein can have a beam spot size that ranges from about 30 microns to about 3 mm, from about 50 microns to about 1 mm, or from about 50 microns to about 500 microns. The pulse width can range from about 1 nanosecond to continuous wave, or from about 1 microsecond to about 100 milliseconds. The fluence can range from about 0.1 J/cm² to about 1000 J/cm².

In one embodiment, the coagulate damage caused by non-ablative fractional treatment is combined with the ablative damage cause by fractional ablative treatment. The combination of non-ablative fractional and ablative fractional offers an opportunity to vary systematically both treatment modalities. The combination of fractional ablative and fractional non-ablative treatments can be employed in a single patient treatment/patient visit to improve cosmetic conditions including, but not limited to: wrinkles, skin tightening, discromia, vascular lesions, pigmented lesions, acne, acne scars, surgical scars, atrophic scars, and stretch marks.

In one embodiment, in a single patient treatment and/or a single patient visit to the practitioner, a non-ablative fractional treatment is combined with an ablative fractional treatment. In one embodiment, the fractional non-ablative portion of the treatment is performed prior to the fractional ablative portion of the treatment. In another embodiment, the fractional ablative treatment is performed prior the fractional non-ablative treatment. The fractional non-ablative treatment can coagulate the treated tissue from the surface down to deep portions of the skin, for example, as deep as the dermal hypodermal junction. The fractional ablative treatment can create voids in the treated tissue such that the voids are created superficially and/or are as deep as about 1 mm in depth. The fractional ablative treatment treats the skin by ablating the treated tissue.

Combining each of the two treatment modalities enables single treatment coverage to extend to a higher percentage of coverage in a single treatment than what is possible with either modality used alone. For example, combining fractional non-ablative treatments with fractional ablative treatments all within a single treatment session as disclosed herein enables results in a single treatment session that would normally take three or four separate non-ablative fractional treatment sessions. In addition, combining fractional non-ablative treatments with fractional ablative treatments all within a single treatment session as disclosed herein enables results in a single treatment session that would normally take three or four or multiple separate ablative fractional treatment sessions. In addition, combining fractional non-ablative treatments with fractional ablative treatments all within a single treatment session as disclosed herein enables results in a single treatment session that would normally take three or four or multiple separate ablative and/or non-ablative fractional treatment sessions. Further, combining the fractional non-ablative treatment with fractional ablative treatments as disclosed herein enables a higher percentage of overall coverage (i.e., percentage of tissue treated) without long term side effects (e.g., hyper pigmentation) then is possible with either treatment modality used alone.

In clinical testing, a fractional non-ablative treatment was combined with a fractional ablative treatment in accordance with the conditions and ranges described in Table 2.

TABLE 2

| | | | Area | | Depth |
|---|---|---|---|---|---|
| Microfractional | | Millijoules | Coverage | | with |
| Handpiece | Type | (MJ) | Range | Depth | PCA |
| Lux2940 ™ | Fractional | 5-24 | 10-30% | Up to | No data |
| | Ablative | | | about | |
| | | | | 1 mm | |
| Lux1440 ™ | Fractional | Up to 12 | 10-30% | 300-400 | No data |
| | non-ablative | | | micron | |
| Lux1540 ™ | Fractional | 50-70 | 10-30% | 1.0 mm | 1.5 mm |
| | non-ablative | | | | |

A fractional non-ablative treatment combines with a fractional ablative treatment to provide area coverage that ranges from about 20% to about 60% (see Table 1). Where each of the individual treatment modalities, fractional ablative and fractional non-ablative, is in the range of 25% to 30% coverage then each individual treatment modality is at or is very close to its own maximum and the area coverage ranges from about 50% to about 60%. Combining the two treatment modalities in this way enables in a single treatment a percentage of coverage called the combined coverage or the overall coverage (e.g., from about 50% to about 60%) that exceeds the maximum coverage for each of the individual treatment modalities. In some instances, the combination provides a maximum area coverage (e.g, 60%) that is double the available coverage of an individual treatment modality (e.g., where the fractional non-ablative maximum is 30%). If either fractional ablative or fractional non-ablative were individually used to provide 50% coverage in a single treatment there would be a high risk of hyper-pigmentation or hypo-pigmentation, however, by combining the two treatments to provide overall coverage of about 50% (25% from non-ablative fractional and 25% from ablative fractional) the risk of hyper-pigmentation or hypo-pigmentation is reduced.

Unlike fractional non-ablative employed alone, when combined with fractional ablative there can be some associated downtime. Generally, the downtime is the sum of the downtimes that would be required for each treatment modality used individually. For example, depending on the depth of treatment where the percentage of coverage ranges from about 10% to about 40% the amount of downtime associated with a fractional ablative procedure ranges from about 2 days to 7 days. The fractional non-ablative portion of the treatment would not add to the associated downtime, accordingly, the downtime would depend only on the depth and the percentage of fractional ablative coverage and be determined no differently than if fractional ablation were used alone. Accordingly, the use of fractional non-ablative treatment in combination with the fractional ablative treatment does not add to the side effect of downtime associated with fraction ablative treatment.

At least some of the benefits of the combination of non-ablative fractional and ablative fractional treatment appear to be additive. For example, relative to fractional ablative used alone the combination with fractional non-ablative provides more density of coverage with less downtime (e.g., with fractional ablative treatment additional percentages of coverage is associated with increased downtime). The combination can enable collagen healing over a treatment area and/or a larger coverage area (e.g., including the non-ablative fractional portions) without as much downtime as is associated with higher coverage fractional ablative treatment.

Without being bound to a single theory, it is believed that use of fractional ablation initiates an intensive tissue healing response and the fractional non-ablative treatment portions take advantage of this healing response as well. The whole body works to heal both ablative and non-ablative treatment areas and the fractional ablative treatment initiates a more intense healing response, e.g. greater erythema and greater edema than is initiated by non-ablative fractional coagulation alone. Healing response refers to the degree of erythema and edema as well as the duration of the erythema and the edema prior to fully healed skin.

Combining fractional ablative treatment with fractional non-ablative treatment as disclosed herein can provide in a single treatment session similar results as previously achieved with fractional CO₂ treatment. The combined fractional ablative and fractional non-ablative treatment requires fewer days of downtime compared to fractional CO₂ treatment. Fractional CO₂ requires from about 6 days to about 7 days of downtime compared with the 4 days to about 5 days of downtime required when combining fractional ablative treatment with fractional non-ablative treatment as disclosed herein.

Use of ablative fractional treatment can enhance the delivery of topical treatments. The wounds created via fractional ablative treatments enable a topical to be delivered to the skin and/or the patient via the wounds. Suitable topical agents include, for example, wound healing compounds that can treat the open wounds created by the fractional ablative treatment. Optionally, the fractional ablative treatment areas may be employed for delivery of filler type compounds (e.g., restyline) into the void spaces that were created during the ablative fractional treatment.

### Example Treatment 1

The effects of non-ablative (e.g., coagulative) and ablative injury and their importance in an immediate skin tightening reaction are difficult to observe during fractional skin resurfacing procedures due to inflammatory skin reactions.

In one embodiment, a human ex vivo tissue model was developed and used to quantitatively examine skin tightening advantages for combining fractional ablative and fractional non-ablative treatments. Parameters based upon results from the model were then used in a clinical study for facial skin rejuvenation. Facial skin from rhytidectomies was treated with fractional ablation using the Palomar® Lux2940™ micro-fractional handpiece the facial skin was also treated with non-ablative fractional treatment using the Palomar® Lux1540™ micro-fractional handpiece and/or the Palomar® Lux1440™ micro-fractional handpiece under controlled temperature and hydration conditions.

Tissue shrinkage was quantified as a function of depth and density of fractional treatment. Safety, side effects, and effectiveness (Fitzpatrick Wrinkle Scale, 0-9, and quartile grading) with a minimum of 3 month follow-up were evaluated in patients (n=18) for facial rejuvenation using several combinations of fractional non-ablative and fractional ablative parameters with combined coverage reaching over 50%.

In the ex vivo model, the percent tissue shrinkage in an area was directly proportional to the density of treatment for both ablative and non-ablative modalities. Contraction in the 3^{rd} dimension was substantial and related to the density and the depth of injury. When combined, the effects of both modalities were additive and combination settings suitable for clinical evaluations were observed to yield up to 30% tissue shrinkage. Patient treatments were well-tolerated with only topical anesthesia. Occasional minimal bleeding and oozing was observed with rapid re-epithelialization. Erythema was longer-lasting than low coverage procedures but resolved with minimal complications using standard topical treatments.

Combination treatments employed approximately 25% fill or coverage for each treatment modality (e.g., 25% coverage with fractional non-ablative and 25% coverage with fractional ablative) of 150 to 300 μm depths consistently resulted in over 75% improvement in skin texture and pigmentation in a single treatment with improved and reduced downtime compared to a high coverage fractional ablative-only procedure. Combined high coverage deeper treatments from 300 μm to 1 mm for target deep periorbital, perioral and cheeks could achieve over 50% reduction in wrinkles with notable reduction in facial laxity with approximately ½ the downtime and discomfort of full-ablative procedures.

Combined fractional non-ablative and fractional ablative treatments made higher single treatment combined coverage (≧50%) possible and resulted in greater effectiveness than either modality alone. Combining these modalities offers new strategies for skin rejuvenation, which helps manage side effects and the downtime associated with fractional ablative-only procedures.

### Example Treatment 2

In another embodiment, A new strategy to combine the coagulate damage from fractional non-ablative treatment with the ablative damage from a fractional ablative treatment was evaluated in an ex vivo model for skin shrinkage and in a clinical study for facial skin rejuvenation.

Facial skin from rhytidectomies was treated with fractional ablation using the Palomar® Lux2940™ micro-fractional handpiece the facial skin was also treated with non-ablative fractional treatment using the Palomar® Lux1540™ micro-fractional handpiece and/or the Palomar® Lux1440™ micro-fractional handpiece under controlled conditions.

Tissue shrinkage was quantified as a function of depth and density of fractional treatment. Safety, side effects, and effectiveness with a minimum of 3 month follow-up visits were evaluated in 18 patients for facial rejuvenation with combined fractional non-ablative and fractional ablative combined coverage reaching over 50%.

Skin tightening was observed in the ex vivo model that was directly proportional to both density and depth of treatment. When combined, the fractional ablative and fractional non-ablative effects were additive yielding up to 30% tissue shrinkage. At similar settings, patient treatments were well-tolerated with only topical anesthesia, had minimal bleeding and re-epithelialized within 4 days.

A single combination treatment employed approximately 25% fill or coverage for each treatment modality (e.g., 25% coverage with fractional non-ablative and 25% coverage with fractional ablative) at <300 μm depths resulted in over 75% improvement in skin texture, fine lines and pigmentation with acceptable side effects and downtime compared to lower coverage fractional ablative-only treatments. Combination treatments with column depths from 300 μm to 1 mm achieved over 50% reduction in wrinkles and notable reduction in facial laxity.

Combined fractional non-ablative and fractional ablative treatments made higher single treatment combined coverage (≧50%) possible and resulted in greater effectiveness than either modality alone. Combining these modalities offers new strategies for skin rejuvenation which helps manage side effects and the downtime associated with fractional ablative-only procedures.

### Example Treatment 3

A 1540 nm fractional non-ablative device employed a point compression array (PCA) optic that enhances the depth of coagulation and reduces epidermal damage. Such deep non-ablative fractional treatments were combined with a groove pattern of fractional ablation using an Er:YAG laser to determine maximum tolerable coverage with acceptable side effects and healing time. The goal was to identify a single treatment strategy to rejuvenate and tighten lax skin on the neck.

The treatments consisted of multiple passes with a 1540 nm laser (i.e., a Palomar® Lux1540™ micro-fractional handpiece) equipped with a point-compression-array optic followed by multiple passes with a Palomar® Lux2940™ micro-fractional handpiece equipped with a groove pattern optic. The orientation of the parallel lines of ablation generated by the groove optic treatment was varied systematically. Subjects (n=12) received a single treatment coverage of 10-30% for each device. Safety, side effects and efficacy were evaluated with investigator assessments and standardized photographic grading.

Histology results from treatment with the Lux1540™ point compression array optics showed coagulation columns of 1.5 mm depths. Lux2940™ treatments with groove optics generated channels up to 50011 depths. Patient treatments were well-tolerated, had minimal bleeding and re-epithelialized within 4 days. Combination treatments with deep fractional coagulation and grooved ablation patterns demonstrated reduction in laxity with very good improvements in dyschromia and skin texture. Results indicate that the combination of non-ablative fractional and ablative-fractional treatments allow higher coverage rates (40-50%) than are possible when either treatment modality is used alone with rapid healing and manageable side effects.

Combined deep dermal coagulation of non-ablative fractional treatment with the groove pattern of ablative fractional treatment resulted in significant rejuvenation and a reduction in skin laxity. Combining these modalities enables new strategies to rejuvenate and tighten non-facial skin.

A disclosed herein, an applicator having one or more point compression element(s) that displace tissue fluid can enhance the depth of coagulation and reduce epidermal damage. By compressing or deforming skin or other tissue in relatively small areas, the electromagnetic radiation (“EMR”), particularly optical radiation such as visible and infrared light, can be delivered to the tissue more effectively. For example, a quantity “A” of EMR can be delivered more deeply into a deformed area of the tissue than if the same area of tissue were not deformed and the quantity “A” of EMR e.g., the same amount of energy, were delivered. This phenomenon, is believed to be particularly applicable to the fractional treatment of tissue with EMR. Use of one or more point compression element(s) provides local deformation of skin tissue using positive or negative pressure to enhance penetration of EMR during the fractional treatment of skin tissue. Without being bound to any single theory, it is believe that skin is compressible, but that tissue fluid (e.g., water) is not. Since skin comprises 60-70% water, then the compressibility of skin arises in part from the displacement of tissue fluid (e.g., water). The deformed tissue accordingly has a modified tissue fluid (e.g., water) content that will change scattering and absorption. In addition to the changes in the optical properties of skin, the thermal properties also may change with compression. For example, with the displacement of water, the average, local thermal properties such as thermal conductivity, specific heat, and thermal diffusivity will also change. These changes may combine to enhance desired thermal effects in compressed tissue arising from photothermolysis. For example, the adiabatic application of radiation to heat tissue to a desired temperature (e.g., for coagulation) will become possible at lower laser power levels where there is a decrease in thermal diffusivity of the tissue (with all other parameters the same).

Referring again to Formulas 1 and 2 disclosed and discussed herein, deformation of tissue (e.g., skin tissue) with one or more point compression element(s) can be altered to achieve desired diffusivity properties. In addition, the change in tissue fluid contact due to deformation can alter the tissue's scattering and absorption properties such that light can penetrate more deeply into the tissue compared to a treatment using the same energy and other parameters but lacking deformation of the tissue via one or more point compression elements.

Referring again to FIGS. 4-6, during treatment (FIG. 5), the contact compression surface of the one or more point compression elements of an applicator 100 are pressed into the surface of an area of tissue, in this case skin. The depth of deformation into the skin can range from about 100 microns to about 3 mm at the deepest point of deformation as compared to the normal surface of the non-deformed skin. The tissue is thereby deformed. While deformed, an array of beams of EMR, each having a diameter (d) and a pitch (l), is applied to the tissue. The array of EMR beams creates an array of treatment zones each having a treatment depth (h). The applicator is then removed from the tissue (FIG. 6), and the tissue relaxes resulting in a treatment depth (H) for each treatment zone. Optionally, one or more of the array of points compression element(s) 110 in the applicator is actively cooled for example, to a temperature of below −5° C. Any of a number of wavelength ranges may be emitted by the fractional beams, for example, the wavelength ranges can include: 1540 nm, 1440 nm, and 1208 nm.

### EQUIVALENTS

While only certain embodiments have been described, it will be understood by those skilled in the art that various changes in form and details may be made therein without departing from the spirit and scope as defined by the appended claims. Those skilled in the art will recognize, or be able to ascertain using no more than routine experimentation, many equivalents to the specific embodiments described specifically herein. Such equivalents are intended to be encompassed in the scope of the appended claims.

### REFERENCES AND DEFINITIONS

The patent, scientific and medical publications referred to herein establish knowledge that was available to those of ordinary skill in the art. The entire disclosures of the issued U.S. patents, published and pending patent applications, and other references cited herein are hereby incorporated by reference.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent or later-developed techniques which would be apparent to one of skill in the art. In addition, in order to more clearly and concisely describe the claimed subject matter, the following definitions are provided for certain terms which are used in the specification and appended claims.

### Numerical Ranges

As used herein, the recitation of a numerical range for a variable is intended to convey that the embodiments may be practiced using any of the values within that range, including the bounds of the range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≧0 and ≦2 if the variable is inherently continuous. Finally, the variable can take multiple values in the range, including any sub-range of values within the cited range.

As used herein, unless specifically indicated otherwise, the word “or” is used in the inclusive sense of “and/or” and not the exclusive sense of “either/or.”

As used herein, EMR includes the range of wavelengths approximately between 200 nm and 10 mm. Optical radiation, i.e., EMR in the spectrum having wavelengths in the range between approximately 200 nm and 100 μm, is employed in some of the embodiments described above, but, also as discussed above, many other wavelengths of energy can be used alone or in combination. The actual spectrum may also include broad-band components, either providing additional treatment benefits or having no effect on treatment. Additionally, the term optical (when used in a term other than term “optical radiation”) applies to the entire EMR spectrum. Other energy may be used to for treatment islets in similar fashion. For example, sources such as ultrasound, photo-acoustic and other sources of energy may also be used to form treatment islets. Thus, although the embodiments described herein are described with regard to the use of EMR to form the islets, other forms of energy to form the islets are within the scope of the invention and the claims.

What is claimed is:

1. An apparatus for performing a treatment on a tissue, comprising:
a) a treatment device configured to deliver electromagnetic radiation, the device having an electromagnetic radiation source; and
b) an applicator for creating point compression of tissue, the electromagnetic radiation source being in communication with the applicator, the applicator having one or more point compression elements, the one or more point compression elements each having a contact compression surface, the applicator being configured to deliver an electromagnetic radiation beam through the contact compression surface during operation, wherein the contact compression surface has a size larger than the electromagnetic radiation beam delivered there through.

2. The apparatus of claim 1 wherein at least a portion of the applicator is cooled.

3. The apparatus of claim 1 wherein at least a portion of the one or more point compression elements is cooled.

4. The apparatus of claim 1 wherein at least a portion of the one or more point compression elements is transparent to electromagnetic radiation.

5. The apparatus of claim 1 wherein the applicator is configured to deliver the electromagnetic radiation beam solely through an inner perimeter of the contact compression surface.

6. The apparatus of claim 1 wherein the one or more point compression elements form an array and the distance between adjacent point compression elements is substantially constant.

7. The apparatus of claim 6 wherein the portion of one contact compression surface that delivers the electromagnetic radiation beam is a substantially constant distance from the portion of an adjacent contact compression surface that delivers the electromagnetic radiation beam.

8. The apparatus of claim 6 wherein the applicator is configured to deliver the electromagnetic radiation beam through the contact compression surface such that the distance between adjacent electromagnetic radiation beams is substantially constant.

9. The apparatus of claim 1 wherein at least one dimension of the contact compression surface of the point compression element is less than about 3 mm.

10. The apparatus of claim 1 wherein the distance between adjacent point compression elements is greater than the two times smallest dimension of the contact compression surface.

11. The apparatus of claim 1 wherein at least one dimension of the contact compression surface is no more than twice the desired depth of tissue treatment.

12. The apparatus of claim 1 wherein the one or more point compression elements each has a collocated electromagnetic radiation beam.

13. The apparatus of claim 12 wherein the distance between adjacent electromagnetic radiation beams is substantially the same.

14. The apparatus of claim 1 further comprising a mechanical implement that applies pressure to the one or more pint compression elements.

15. The apparatus of claim 1 further comprising a transducer that provides a signal when a desired compression of tissue is reached.

16. The apparatus of claim 11 wherein the desired compression of tissue is up to 50 Newtons/cm².

17. The apparatus of claim 1 further comprising a source of electromagnetic radiation with a wavelength range of from about 290 nm to about 11000 nm, a fluence of from about 0.1 J/cm² to about 1000 J/cm², a pulse width of from about 1 nanosecond to continuous wave, and a radiation beam with a spot size of from about 50 microns to about 3 mm.

18. A method for treating a region of tissue, comprising:
a) pressing a contact compression surface of one or more point compression elements to a skin surface; and
b) applying electromagnetic radiation through the one or more contact compression surfaces, wherein each of the one or more contact compression surfaces has a size larger than the size of the electromagnetic radiation beam delivered there through.

19. The method of claim 18 wherein the electromagnetic radiation beam causes at least one of thermal, chemical, and mechanical effects on the region of tissue.

20. The method of claim 18 further comprising applying two or more pulses of electromagnetic radiation without removing the one or more point compression elements from the skin surface between applied pulses.

21. The method of claim 18 wherein step a further comprises:
a) pressing a contact compression surface of one or more point compression elements to a skin surface to displace tissue fluid from the compressed tissue.

22. The method of claim 21 wherein the tissue fluid comprises at least one of water, blood, and lymph.

23. The method of claim 21 wherein step b further comprises:
b) applying electromagnetic radiation through the one or more contact compression surfaces, wherein each of the one or more contact compression surfaces has a size larger than the size of the electromagnetic radiation beam delivered there through and wherein the electromagnetic radiation is delivered to at least a portion of the compressed tissue where tissue fluid is displaced due to compression by the contact compression surface of the point compression element.

24. The method of claim 18 wherein step a further comprises:
a) pressing a contact compression surface of one or more point compression elements to a skin surface for at least 1 second.

25. The method of claim 18 further comprising cooling at least a portion of the skin surface with one or more point compression elements.

26. The method of claim 18 wherein step b further comprises:
b) applying electromagnetic radiation through the one or more contact compression surfaces, the electromagnetic radiation source is in communication with an applicator, and the one or more point compression elements are disposed on the applicator, wherein each of the one or more contact compression surfaces has a size larger than the size of the electromagnetic radiation beam delivered there through.

27. The method of claim 18 further comprising cooling at least a portion of the skin surface with the applicator.

28. The method of claim 18 wherein at least one dimension of the one or more contact compression surface is no more than three times greater than the desired depth of tissue treatment.

29. The method of claim 28 wherein the treatment depth ranges from about 0.1 mm to about 10 mm.

30. The method of claim 28 wherein the treatment depth ranges from about 0.1 mm to about 3 mm.

31. The method of claim 18 wherein the surface area of the one or more contact compression surfaces is no more than 30% of the surface of the region of tissue being treated.

32. The method of claim 18 wherein at least one dimension of the one or more contact compression surface is no more than the desired depth of tissue treatment.

33. The method of claim 18 wherein at least a portion of each of the one or more point compression elements is transparent to electromagnetic radiation.

34. The method of claim 18 wherein the one or more point compression elements form an array and the distance between the center of adjacent point compression elements is substantially constant and step b further comprises:
b) applying electromagnetic radiation through the center of the one or more contact compression surfaces such that the distance between adjacent electromagnetic radiation beams is substantially constant, wherein each of the one or more contact compression surfaces has a size larger than the size of the electromagnetic radiation beams delivered there through.

35. The method of claim 18 wherein the one or more point compression elements each has a collocated electromagnetic radiation beam.

36. The method of claim 18 wherein the distance between adjacent electromagnetic radiation beams is substantially the same.

37. The method of claim 18 further comprising applying electromagnetic radiation with a wavelength range of from about 290 nm to about 11000 nm, a fluence of from about 0.1 J/cm2 to about 1000 J/cm2, a pulse width of from about 1 nanosecond to continuous wave, and a radiation beam with a spot size of from about 50 microns to about 3 mm.

38. The method of claim 18 wherein the one or more point compression elements have a depth of deformation into the region of tissue that ranges from about 100 microns to about 3 mm at the deepest point of deformation relative to the normal surface of the skin surface.

39. The method of claim 18 further comprising:
c) receiving a signal when a desired compression is reached.

40. The method of claim 39 wherein the desired compression is up to 50 Newtons/cm².
